(19) United States
(12) Patent Application Publication
(10) Pub. No.: US 2002/0155138 A1
(43) Pub. Date: Oct. 24, 2002

(54) Composition containing Hedychium extract and use thereof

(76) Inventors: Katharine M. Martin, Ringoes, NJ (US); Claude Saliou, Gladstone, NJ (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/052,315

(22) Filed: Jan. 18, 2002

Related U.S. Application Data
(60) Provisional application No. 60/262,822, filed on Jan. 19, 2001.

(51) Int. Cl. A61K 35/78; A61K 7/06

(52) U.S. Cl. 424/401; 424/725; 424/74

(57) ABSTRACT

The present invention features a composition for regulating the firmness, tone, or texture of skin, or for regulating wrinkles, or for the treatment of environmental damage in skin containing a safe and effective amount of a Hedychium extract and a cosmetically-acceptable carrier, and the use thereof.

### CROSS-REFERENCE

This application claims priority from provisional application Ser. No. 60/262,822 filed Jan. 19, 2001.

### FIELD OF THE INVENTION

The present invention relates to compositions comprising Hedychium extract and the cosmetic use thereof.

### BACKGROUND OF THE INVENTION

Plants from the Hedychium genus are perennial rhizomatus plants belonging to the Zingiberaceae family. In particular, the species Hedychium spicatum grows naturally in subtropical regions such as India and China, where it is used as a flowering ornamental and as a traditional medicine. It is also cultivated in various parts of the world for its fragrant rhizome. Traditional indications of Hedychium spicatum include stomactic, indigestion, calmative, bitter tonic, stimulant for dyspepsia, expectorant, liver disorders, hair growth promotor, anti-bacterial, anti-fungic, and anti-malaria. See, X. Yan, et al., Traditional Chinese Medicines, Molecular Structures, Natural Sources, and Applications. (Ashgate Publishing Co., Burlington, Vt., 1999); and Sharma et al., Phytochemistry 14:578 (3975).

### SUMMARY OF THE INVENTION

In one aspect, the invention features a composition for regulating the firmness, tone, or texture of skin or regulating wrinkles in skin containing a safe and effective amount of a Hedychium extract and a cosmetically-acceptable carrier. In another aspect, the invention features a composition for the treatment of environmental damage in skin including a safe and effective amount of a Hedychium extract and a cosmetically-acceptable carrier. In another aspect, the present invention also features the use of such compositions.

Other features and advantages of the present invention will be apparent from the detailed description of the invention and from the claims

### DETAILED DESCRIPTION OF THE INVENTION

It is believed that one skilled in the art can, based upon the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference.

As used herein, “topical application” means directly laying on or spreading on outer skin using, e.g., by use of the hands or an applicator such as a wipe.

As used herein, “cosmetically-acceptable” means that the extracts, drugs, medicaments or inert ingredients which the term describes are suitable for use in contact with tissues (e.g., the skin) without undue toxicity, incompatibility, instability, irritation, allergic response, and the like, commensurate with a reasonable benefit/risk ratio.

As used herein, “regulating the firmness of skin” means the enhancing of the firmness or elasticity of the skin, preventing the loss of firmness or elasticity of skin, or preventing or treating sagging, lax and loose skin. The firmness or elasticity of the skin can be measured by use of a cutometer. See Handbook of Non-Invasive Methods and the Skin, eds. J. Serup & G. Jemec, Chapter 14.3 (1995). The loss of skin elasticity or firmness may be a result of a number of factors, including but not limited to aging, environmental damage, or the result of an application of a cosmetic to the skin.

As used herein, “regulating the tone of skin” means the lightening and/or darkening the skin (e.g., lightening pigmented lesions or darkening skin sallowness).

As used herein, “regulating the texture of skin” means the smoothing of the surface of the skin to remove either bumps or crevasses on the skin surface.

As used herein, “regulating wrinkles in skin” means preventing, retarding, arresting, or reversing the process of wrinkle and fine line formation in skin.

As used herein, “treatment of environmental damage in skin” means the reduction or prevention in the effects of environmental damage in skin. Examples of environmental damage include, but are not limited to, damage from the UV radiation (e.g., from the sun or non-natural sources such as UV lamps and solar simulators), ozone, exhaust, pollution, and cigarette smoke. Effects of environmental damage on the skin include, but are not limited to, oxidative and/or nitrosative damage to and modifications on lipids, carbohydrates, peptides, proteins, nucleic acids, and vitamins. Effects of environmental damage on the skin also include, but are not limited to, loss of cell viability, loss or alteration of cell functions, and changes in gene and/or protein expression.

As used herein, “safe and effective amount” means an amount of compound or composition (e.g., the Hedychium extract) sufficient to significantly induce a positive modification in the condition to be regulated or treated, but low enough to avoid serious side effects (at a reasonable benefit/risk ratio), within the scope of sound medical judgment. The safe and effective amount of the compound or composition will vary with the particular condition being treated, the age and physical condition of the end user, the severity of the condition being treated/prevented, the duration of the treatment, the nature of concurrent therapy, the specific compound or composition employed, the particular cosmetically-acceptable carrier utilized, and like factors.

As used herein, all percentages are by weight unless otherwise specified.

What is meant by a “Hedychium extract” is a blend of compounds isolated from a plant from the Hedychium genus (e.g., the Hedychium spicatum plant). Such compounds may be isolated from a part(s) of the plant (e.g., the seed, root, rhizome, fruit and/or leaf of the plant) by physically removing a piece of such plant, such as grinding a leaf on the plant. Such compounds may also be isolated from the plant by using extraction procedures well known in the art (e.g., the use of organic solvents such as lower C₁-C₈ alcohols, C₁-C₈ alkyl polyols, C₁-C₈ alkyl ketones, C₁-C₈ alkyl ethers, acetic acid C₁-C₈ alkyl esters, and chloroform, and/or inorganic solvents such as water, inorganic acids such as hydrochloric acid, and inorganic bases such as sodium hydroxide). In one embodiment, the Hedychium extract contains only hydrophilic compounds (e.g., isolated by using a hydrophilic solvent, such as water or ethanol). In one embodiment, the Hedychium extract contains only hydrophobic compounds (e.g. isolated by using a hydrophobic solvent, such as chloroform). In one embodiment, the Hedychium extract contains both hydrophilic and hydrophobic compounds.

Examples of plants from the Hedychium genus include, but are not limited to, Hedychium Spicatum and Hedychium Coronarium, as well as those listed in CRC Ethnobotany Desk Reference 1998, ed. Timothy Johnson, p 394 (CRC Press, Boca Raton, Fla., USA 1998) and the ‘The Plant Names Project (1999). International Plant Names,Index. Published on the Internet; http://www.ipni.org [accessed Jan. 11, 2001].

The amount of the Hedychium extract present in the composition will depend on the type of extract used. The extract typically will be present in the composition in an amount from about 0.001% to about 20% by weight, in particular in an amount from about 0.01% to about 1% by weight.

The topical compositions useful in the present invention involve formulations suitable for topical application to skin. The compositions may be made into a wide variety of product types that include but are not limited to lotions, creams, gels, sticks, sprays, ointments, cleansing liquid washes and solid bars, shampoos, pastes, mousses, wipes, patches, wound dressing and adhesive bandages, hydrogels, films-and cosmetics. These product types may comprise several types of cosmetically acceptable carrier systems including, but not limited to solutions, emulsions, gels, solids and liposomes.

The topical compositions useful in the present invention can be formulated as solutions. Solutions typically include an aqueous or organic solvent (e.g., from about 80% to about 99.99% or from about 90% to about 99% of an acceptable aqueous or organic solvent). Examples of suitable organic solvents include: propylene glycol, polyethylene glycol (200-600), polypropylene glycol (425-2025), glycerol, 1,2,4-butanetriol, sorbitol esters, 1,2,6-hexanetriol, ethanol, and mixtures thereof.

Topical compositions useful in the subject invention may be formulated as a solution comprising an emollient. Such compositions preferably contain from about 2% to about 50% of an emollient(s). As used herein, “emollients” refer to materials used for the prevention or relief of dryness, as well as for the protection of the skin. A wide variety of suitable emollients are known and may be used herein. Sagarin, Cosmetics, Science and Technology, 2nd Edition, Vol. 1, pp. 32-43 (1972) and the International Cosmetic Ingredient Dictionary and Handbook, eds. Wenninger and McEwen, pp. 1656-61, 1626, and 1654-55 (The Cosmetic, Toiletry, and Fragrance Assoc., Washington, D.C., 7^{th} Edition, 1997) (hereinafter “ICI Handbook”) contains numerous examples of suitable materials

A lotion can be made from such a solution carrier system. Lotions typically comprise from about 1% to about 20% (e.g., from about 5% to about 10%) of an emollient(s) and from about 50% to about 90% (e.g., from about 60% to about 80%) of water.

Another type of product that may be formulated from such a solution carrier system is a cream. A cream typically comprises from about 5% to about 50% (e.g., from about 10% to about 20%) of an emollient(s) and from about 45% to about 85% (e.g., from about 50% to about 75%) of water.

Yet another type of product that may be formulated from such a solution carrier system is an ointment. An ointment may comprise a simple base of animal or vegetable oils or semi-solid hydrocarbons. Ointments may also comprise absorption ointment bases that absorb water to form emulsions. An ointment may comprise from about 2% to about 10% of an emollient(s) plus from about 0.1% to about 2% of a thickening agent(s). A more complete disclosure of thickening agents or viscosity increasing agents useful herein can be found in Sagarin, Cosmetics, Science and Technology, 2nd Edition, Vol. 1, pp. 72-73 (1972) and the ICI Handbook pp. 1693-1697.

The topical compositions useful in the present invention formulated as emulsions. If the carrier is formulated as an emulsion, from about 1% to about 10% (e.g., from about 2% to about 5%) of the carrier system comprises an emulsifier(s). Emulsifiers may be nonionic, anionic or cationic. Suitable emulsifiers are disclosed in, for example, U.S. Pat. No. 3,755,560, U.S. Pat. No. 4,421,769, McCutcheon's Detergents and Emulsifiers, North American Edition, pp. 317-324 (1986), and the ICI Handbook, pp. 1673-1686.

Lotions and creams can be formulated as emulsions. Typically such lotions comprise from 0.5% to about 5% of an emulsifier(s). Such creams would typically comprise from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion skin care preparations, such as lotions and creams, of the oil-in-water type and water-in-oil type are well-known in the cosmetic art and are useful in the subject invention. Multiphase emulsion compositions, such as the water-in-oil-in-water type, as disclosed in U.S. Pat. Nos. 4,254,105 and 4,960,764, are also useful in the subject invention. In general, such single or multiphase emulsions contain water, emollients, and emulsifiers as essential ingredients.

The topical compositions of the present invention can also be formulated as a gel (e.g., an aqueous, alcohol, alcohol/water or oil gel using a suitable gelling agent (s)). Suitable gelling agents for aqueous and/or alcoholic gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically comprises between about 0.1% and 5%, by weight, of such gelling agents.

The topical compositions of the present invention can also be formulated into a solid formulation (e.g., a wax-based stick or soap bar composition).

Liposomal formulations are also useful compositions of the subject invention. Such compositions can be prepared by first combining hesperetin with a phospholipid, such as dipalmitoylphosphatidyl choline, cholesterol and water according to the method described in Mezei & Gulasekharam, “Liposomes—A Selective Drug Delivery System for the Topical Route of Administration; Gel Dosage Form”, Journal of Pharmaceutics and Pharmacology, Vol. 34 (1982), pp. 473-474, or a modification thereof. Epidermal lipids of suitable composition for forming liposomes may be substituted for the phospholipid. The liposome preparation is then incorporated into one of the above topical carrier systems (e.g., a gel or an oil-in-water emulsion) in order to produce the liposomal formulation. Other compositions and pharmaceutical uses of topically applied liposomes are described in Mezei, M., “Liposomes as a Skin Drug Delivery System”, Topics in Pharmaceutical Sciences (D. D. Breimer and P. Speiser, eds.,), Elsevier Science Publishers B.V., New York, N.Y., 1985, pp. 345-358, incorporated herein by reference.

The topical compositions useful in the subject invention may contain, in addition to the aforementioned components, a wide variety of additional oil-soluble materials and/or water-soluble materials conventionally used in topical compositions, at their art-established levels.

In one embodiment, the topical composition further comprises another cosmetically active agent in addition to the Hedychium extract. What is meant by a “cosmetically active agent” is a compound that has a cosmetic or therapeutic effect on the skin, e.g., lightening agents, anti-acne agents, anti-bacterial agents, anti-mycotic agents, anti-parasite agents, external analgesics, sunscreens, photoprotectors, antioxidants, keratolytic agents, detergents/surfactants, moisturizers, nutrients, energy enhancers, anti-perspiration agents, astringents, deodorants, hair removers, and agents for hair and/or skin conditioning.

In one embodiment, the agent is selected from, but not limited to, the group consisting of hydroxy acids, benzoyl peroxide, sulfur resorcinol, ascorbic acid, D-panthenol, hydroquinone, sunscreen agents, anti-inflammatory agents, skin lightening agents, antimicrobial and antifungal agents, vitamins, polyphenolics, carotenoids, free radical scavengers, spin traps, retinoids such as retinol and retinyl palmitate, ceramides, polyunsaturated fatty acids, essential fatty acids, enzymes, enzyme inhibitors, minerals, estrogens, 2-dimethylaminoethanol, copper peptides such as Cu:GHK, lipoic acid, amino acids such a proline and tyrosine, lactobionic acid, acetyl-coenzyme A, niacin, riboflavin, thiamin, ribose, electron transporters such as NADH and FADH2; and botanical extracts such as aloe vera and soy, and derivatives and mixtures thereof. The cosmetically active agent will typically be present in the composition of the invention in an amount of from about 0.001% to about 20% by weight of the composition, e.g., about 0.01% to about 10% such as about 0.1% to about 5%.

Examples of hydroxy acids include, but are not limited, to (i) alpha-hydroxy acids such as glycolic acid, lactic acid, malic acid, citric acid, and tartaric acid, (ii) beta-hydroxy acids such as salicylic acid, and/or (iii) polyhydroxy acids. See, e.g., European Patent Application No. 273,202.

Examples of derivatives of ascorbic acid include, but are not limited to, ascorbyl palmitate, magnesium ascorbyl phosphate, sodium ascorbyl phosphate, zinc ascorbyl phosphate, ascorbyl glucoside, sodium ascorbate, and ascorbyl polypeptide. An example of a derivative of hydroquinone includes, but is not limited to, arbutin.

Various water-soluble materials may also be present in the compositions useful in the subject invention. These, include humectants, proteins and polypeptides, preservatives and an alkaline agent. Examples of such agents are disclosed in the ICI Handbook, pp. 1650-1667. The compositions of the present invention may also comprise administering a composition containing one or more of the following: antioxidants (e.g., ascorbic acid, tocopherols, BHA, polyphenols, carotenoids, alpha-lipoic acid, glutathione precursors, tocotrienols, iron chelators and BHT), chelating agents (e.g., EDTA), and preservatives (e.g., parabens). Examples of suitable antioxidants, preservatives, and chelating agents are listed in pp. 1612-13, 1626, and 1654-55 of the ICI Handbook. In addition, the topical compositions useful herein can contain conventional cosmetic adjuvants, such as dyes, opacifiers (e.g., titanium dioxide), pigments, and fragrances.

The composition and formulations containing such compositions of the present invention may be prepared using methodology that is well known by an artisan of ordinary skill.

### EXAMPLE 1

### Inhibition of UV Induced MMP

The ability of Hedychium spicatum to inhibit UV induced matrix metalloproteinase-1 (MMP-1) was evaluated in epidermal equivalents derived from normal human epidermal keratinocytes. MMPs are a family of enzymes that play a major role in physiological remodeling and pathological destruction of extracellular matrix. It is well established that suberythemal doses of UV light induce MMP secretion in human skin, which in turn degrades the extracellular matrix and play a significant role in photoaging wrinkle formation and loss of firmness and elasticity. See G. J. Fisher, et al., Nature 379:335-339(1996) and G. J. Fisher and J. J. Voorhees, J. Invest. Dermatol. Symposium Proceedings. 3:61-68 (1998).

In order to evaluate the ability of Hedychium spicatum to inhibit UV induced MMP-1, epidermal equivalents were obtained from SkinEthic (Nice, France) and cultured in phenol free, hydrocortisone free medium (SkinEthic). The equivalents were then topically treated with 0% or 0.5%, by weight, of Hedychium spicatum extract (sold as Kapur Kachari from Amsar P. Ltd., Indore, India) for 1 to 2 hours prior to irradiating with solar spectrum light at doses of 0, 5, 7, 9 and 15 MED using a 1000 Watt solar ultraviolet simulator (Oriel, Stratford, Conn., USA). Forty-eight hours post-irradiation, the medium below each equivalent was then collected and analyzed for secreted MMP-1 by ELSA (Calbiochem, San Diego, Calif., USA). The results of such experiment are set forth in Table 1.

TABLE 1

MMP-1 (pg/ml)

| UV Light (MED) | 0% Hedychium | 0.5% Hedychium |
|---|---|---|
| 0 | 19.3 ± 2.12 | 31.2 ± 14.28 |
| 5 | 28.7 ± 11.56 | 6.85 ± 3.6 |
| 7 | 33.0 ± 4.2 | 5.8 ± 0.07 |
| 9 | 44.0 ± 7.9 | 7.5 ± 1.7 |
| 11 | 28.4 ± 10.0 | 3.7 ± 0.4 |
| 15 | 11.2 ± 1.6 | 2.1 ± 0.6 |

These results indicate that the formulation containing Hedychium spicatum extract was able to provide protection against induction of MMP-1. following irradiation with solar spectrum light up to doses of 15 MED.

### EXAMPLE 2

### Prevention of Smoke-induced Loss of Thiols

The ability of Hedychium spicatum extract to prevent smoke-induced loss of thiols was evaluated in normal human dermal fibroblasts (Clonetics, San Diego, Calif.). Thiols, chiefly glutathione, are part of the endogenous cellular antioxidant defense system. Glutathione serves as a redox buffer, thereby, maintaining the balance between oxidants and antioxidants. Glutathione is also the preferred substrate for several enzymes such as the glutathione peroxidases (decomposing peroxides) and the glutathione-S-transferases (a major group of detoxification enzymes). See, A. Master, Cancer Res. 54:1969s-1975s (1994).

Cutaneous antioxidants (both enzymatic and non-enzymatic), including glutathione, are depleted after UV or ozone exposure. See, M. J. Connor and L. A. Wheeler, Photochem. Photobiol. 46:239-246 (1987) and R. M. Tyrrell and M. Pidoux, Photochem. Photobiol. 47.405-412 (1988). In cell culture models, low intracellular glutathione (GSH) levels lead co a higher UVR sensitivity. Topical application of cysteine derivatives on rat skin has been shown to protect against UVR-induced photodamage; this benefit was correlated with an increase in GSH synthesis. See, L. T. van den Broeke and G. M. J. Beijersbergen van Henegouwen, J. Photochem. Photobiol. B Biol. 27:61-65 (1995); K. Hanada, et al., J. Invest. Dermatol. 108:727-730 (1997); and D. P. T. Steenvoorden, et al., Photochem Photobiol. 67:651-656 (1998). Consequently, glucothione is a major endogenous antioxidant, highly responsive against environmental challenges, able to regulate the tone and the wrinkling of skin, as well as treat environmental damage.

In this experiment, normal human neonatal dermal fibroblasts seeded in 6-well plates transwell inserts were incubated with formulation containing various concentrations of Hedychium spicatum extract for 24 hours Prior to exposure with either placebo (mock) or cigarette smoke for 10 minutes. Prior to smoke exposure, the medium containing the Hedychium spicatum extract was removed and the cells were washed 3 times with phosphate buffered saline pH 7.2 (life Technologies, Gaithersburg, Md.). Immediately after exposure, the cells were incubated for another 2L4-hour period with the previous medium. Intracellular thiols were then measured by adding 60 μM monobromobimane (Molecular Probes, Eugene, Oreg., USA) to the cells for 30 minutes before the fluorescence reading. In zone presence of thiols, the monobromobimane becomes fluorescent. This fluorescence was measured using a CytoFluor® Fluorescence Plate Reader (PerSeptive Biosystems, Framingham, Mass., USA) set with the following filter-combination: excitation at 360 nm and emission at 460 nm.

The results of this experiment are set-forth in Table 2

TABLE 2

| | | Thiols (Percent |
|---|---|---|
| | Hedychium | of Thiols |
| | spicatum extract | contained in No |
| | concentration | Smoke Group; |
| | (μg/ml) | Mean ± S D) |
| No Smoke | 0 | 100 ± 11.99 |
| Smoke (10 min.) | 0 | 65.10 ± 7.93 |
| | 1 | 58.61 ± 10.95 |
| | 10 | 87.07 ± 24.08 |
| | 100 | 106.78 ± 15.84 |

These results indicate that a Hedychium spicatum extract afforded a protection against smoke-induced loss of thiols (data represent 4 to 8 replicates from 2 independent experiment).

### EXAMPLE 3

### Inhibition of Nitric oxide Production

The ability of Hedydchium spicatum extract to inhibit nitric oxide production was evaluated in LPS-stimulated murine macrophages. Nitric oxide is a transducing molecule that has been demonstrated to be involved in physiological processes such as vasodilatation and neurotransmission as well as in pathological processes such as inflammation and cancer. Higher NO levels have been found in psoriasis. It is also well established that high NO concentration for the tissues. In fact, when one NO molecule combines with one superoxide radical, it forms peroxynitrite, a highly toxic free radical species. Applications for materials that decrease NO levels include the following, but are not limited to: regulating the redness and tone of the skin, reducing inflamed skin and vasodilatation, and treatment and prevention of wrinkles and environmental damage in the skin.

The murine macrophages RAW 264.7 (ATCC, Manassas, Va., USA) are co-treated with test-materials and lipopolysaccharides from E. coli. After an 18 hour-incubation period, nitrites released in the medium are measured (nitrite is the immediate down-product in NO metabolism) using the Griess assay. See, Titheradge, Nitric Oxide Protocols, in Methods in Molecular Biology, Vol. 100, pp. 83-91 (Human Press, Totowa, N.J., 1988). Quercetin, a flavonoid known to inhibit NO production is used as a positive control (Sigma Chemicals, Saint Louis, Mo. USA). Hedychium spicatum extract was screened in a concentration range from 10 to 200 μg/ml. The results of this experiment are set forth in the Table 3 below.

TABLE 3

| | Hedychium spicatum | |
|---|---|---|
| LPS | extract | Nitrite |
| Concentration | Concentration | concentration |
| (ng/ml) | (μg/ml) | (μM) |
| 0 | 0 | 4.132 |
| | 10 | 3.278 |
| | 50 | 5.224 |
| | 100 | 4.794 |
| | 200 | 5.339 |
| 100 | 0 | 9.585 |
| | 10 | 10.17 |
| | 50 | 8.365 |
| | 100 | 6.615 |
| | 200 | 5.803 |

Hedychium spicatum extract was found to be an effective inhibitor of NO production, having an IC₅₀ of about 69.97 μg/ml.

It is understood that while the invention has been described in conjunction with the detailed description thereof, that the foregoing description is intended to illustrate and not limit the scope of the invention, which is defined by the scope of the appended claims. Other aspects, advantages, and modifications are within the claims.

What is claimed is:

1. A composition for regulating the firmness, tone, or texture of skin or regulating wrinkles in skin comprising:
(a) a safe and effective amount of a Hedychium extract; and
(b) a cosmetically-acceptable carrier.

2. A composition of claim 1, wherein said composition is for regulating the firmness of skin.

3. A composition of claim 1, wherein said composition is for regulating the tone of skin.

4. A composition of claim 1, wherein said composition is for regulating the texture of skin.

5. A composition of claim 1, wherein said composition is for regulating wrinkles in skin.

6. A composition for the treatment of environmental damage in skin comprising:
(a) a safe and effective amount of a Hedychium extract; and
(b), a cosmetically-acceptable carrier.

7. A composition of claim 6, wherein said environmental damage is damage caused by smoke.

8. A composition of claim 6, wherein said environmental damage is damage caused by UV light.

9. A composition of claim 1, wherein said composition comprises from about 0.001%, by weight, to about 20%, by weight, of said Hedychium extract and said cosmetically-acceptable carrier is a topical carrier.

10. A composition of claim 2, wherein said composition comprises from about 0.001%, by weight, to about 20%, by weight, of said Hedychium extract and said cosmetically-acceptable carrier is a topical carrier.

11. A composition of claim 3, wherein said composition comprises from about 0.001%, by weight, to about 20%, by weight, of said Hedychium extract and said cosmetically-acceptable carrier is a topical carrier.

12. A composition of claim 4, wherein said composition comprises from about 0.001%, by weight, to about 20%, by weight, of said Hedychium extract and said cosmetically-acceptable carrier is a topical carrier.

13. A composition of claim 5, wherein said composition comprises from about 0.001%, by weight, to about 20%, by weight, of said Hedychium extract and said. cosmetically-acceptable carrier is a topical carrier.

14. A composition of claim 6, wherein said composition comprises from about 0.001%, by weight, to about 20%, by weight, of said Hedychium extract and said cosmetically-acceptable carrier is a topical carrier.

15. A composition of claim 7, wherein said composition comprises from about 0.001%, by weight, to about 20%, by weight, of said Hedychium extract and said cosmetically-acceptable carrier is a topical carrier.

16. A composition of claim 8, wherein said composition comprises from about 0.001%, by weight, to about 20%, by weight, of said Hedychium extract and said cosmetically-acceptable carrier is a topical carrier.

17. A method of regulating the firmness, tone, or texture of skin of a subject or regulating wrinkles in skin of a subject, said method comprising the topical administration to said subject a composition comprising:
(a) a safe and effective amount of a Hedychium extract; and
(b) a cosmetically-acceptable carrier.

18. A method of treating environmental damage of skin of a subject, said method comprising the topical administration to said subject a composition comprising:
(a) a safe and effective amount of a Hedychium extract; and
(b) a cosmetically-acceptable carrier.

19. A method of claim 17, wherein said composition comprises from about 0.001%, by weight, to about 20%, by weight, of said Hedychium extract.

20. A method of claim 18, wherein said composition comprises from about 0.001%, by weight, to about 20%, by weight, of said Hedychium extract.
